(19) United States
(12) Patent Application Publication
(10) Pub. No.: US 2026/0250635 A1
(43) Pub. Date: Aug. 27, 2026

(54) TRANSDUCTION AIDS AND TRANSDUCTION METHODS

(71) Applicant: REGENTS OF THE UNIVERSITY OF MINNESOTA, Minneapolis, MN (US)

(72) Inventors: Martin Felices, Circle Pines, MN (US); Jeffrey S. Miller, North Oaks, MN (US); Rih-Sheng Huang, Minneapolis, MN (US)

(21) Appl. No.: 19/550,971

(22) Filed: Feb. 26, 2026

Related U.S. Application Data
(60) Provisional application No. 63/764,186, filed on Feb. 27, 2025.

(51) Int. Cl. C12N 5/0783; C12N 15/86

(52) U.S. Cl. CPC C12N 5/0646; C12N 15/86; C12N 2500/50; C12N 2501/727; C12N 2510/00; C12N 2740/15043; C12N 2760/20243

(57) ABSTRACT

Transduction aids such as polymers, enzyme inhibitors, cytokines, exogenous DNA components, or any combination thereof. Transduction compositions that include one or more transduction aids. Transduction methods that include the use of a transduction composition that include one or more transduction aids.

### CROSS-REFERENCE TO RELATED APPLICATION

This application claims the benefit of U.S. Provisional Patent Application No. 63/764,186, filed Feb. 27, 2025, which is incorporated herein by reference in its entirety.

### GOVERNMENT FUNDING

This invention was made with government support under CA283892 awarded by the National Institutes of Health. The government has certain rights in the invention.

### SUMMARY

This disclosure describes, in one aspect, a transduction composition. The transduction composition includes one or more transduction aids. In one or more embodiments, the transduction composition includes a poloxamer and a TBK1 inhibitor, a TAK1 inhibitor, or both.

In one or more embodiments, the poloxamer and the TBK1 inhibitor are provided in an amount effective to increase the transduction efficiency of a viral vector into a cell by 1-fold or greater compared to the same transduction composition lacking the poloxamer and the TBK1 inhibitor.

In one or more embodiments, the poloxamer includes poloxamer F-108, poloxamer 407, or poloxamer 338. In one or more embodiments, the TBK1 inhibitor includes BX795, MRT67307, BAY-985, or GSK8612. In one or more embodiments, the TAK1 inhibitor comprises 5Z-7-oxozeaenol, NG25, or takinib. In one or more embodiments, the poloxamer includes poloxamer F-108 and the TBK1 inhibitor includes BX795.

In one or more embodiments, the transduction composition includes 0.1 mg/mL to 10 mg/mL of the poloxamer. In one or more embodiments, the transduction composition includes 1 μM to 8 μM of the TBK1 inhibitor or TAK1 inhibitor.

In one or more embodiments, the transduction composition further includes a viral vector. In one or more embodiments, the viral vector comprises a lentiviral vector. In one or more embodiments, the viral vector is a VSV-G pseudotyped vector.

In one or more embodiments, the viral vector includes a CMV promoter, an EFS promoter, an EF1a promoter, an MND promoter, a PGK promoter, an RSV promoter, an SFFV promoter, a CAG promoter, or a UBC promoter.

In one or more embodiments, the transduction composition further includes a cytokine. In one or more embodiments, the cytokine comprises IL-2, IL-12, IL-15, IL-18, IL-21, or any combination thereof.

In one or more embodiments, the transduction composition further includes a medium.

In one or more embodiments, the transduction composition further includes a cell. In one or more embodiments, the cell is a lymphocyte. In one or more embodiments, the cell is a natural killer cell.

In another aspect, the present disclosure describes a transduction method. The transduction method includes contacting a cell with a transduction composition disclosed herein. In one or more embodiments, the cell is a lymphocyte. In one or more embodiments, the cell is a natural killer cell.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 shows the workflow of an experimental procedure. Enriched NK cells were isolated from PBMC and allowed to expand with irradiated mbIL21-41BBL K562 feeder cells for 7 days before transduction. Transduction was performed with small molecules (transduction aids) present in the medium for 24 hours. After 24 hours, the medium was refreshed, and NK cells were cultured for another 72 hours before flow cytometry analysis.

The construct design for CAR-NK generation is shown. The second-generation lentiviral vector contains a self-inactivating (SIN) HIV genome. The vector encodes anti-CD19 CAR with mNeonGreen reporter driven by SFFV promoter. The size of the lentiviral genome is 5073 bp.

FIG. 2 shows plots of the percent mNeonGreen positive cells and relative cell recovery after cells underwent transduction according to workflow of FIG. 1 with polybrene as a transduction aid.

FIG. 3 shows plots of the percent mNeonGreen positive cells and relative cell recovery after cells underwent transduction according to workflow of FIG. 1 with protamine sulfate as a transduction aid.

FIG. 4 shows plots of the percent mNeonGreen positive cells and relative cell recovery after cells underwent transduction according to workflow of FIG. 1 with DEAE-dextran as a transduction aid.

FIG. 5 shows plots of the percent mNeonGreen positive cells and relative cell recovery after cells underwent transduction according to workflow of FIG. 1 with dNs as a transduction aid.

FIG. 6 shows plots of the percent mNeonGreen positive cells and relative cell recovery after cells underwent transduction according to workflow of FIG. 1 with poloxamer F-108 as a transduction aid.

FIG. 7 shows plots of the percent mNeonGreen positive cells and relative cell recovery after cells underwent transduction according to workflow of FIG. 1 with poloxamer 407 as a transduction aid.

FIG. 8 shows plots of the percent mNeonGreen positive cells and relative cell recovery after cells underwent transduction according to workflow of FIG. 1 with poloxamer 338 as a transduction aid.

FIG. 9 shows plots of the percent mNeonGreen positive cells and relative cell recovery after cells underwent transduction according to workflow of FIG. 1 with TBK1/IKKε inhibitor BX795 as a transduction aid.

FIG. 10 shows plots of the percent mNeonGreen positive cells and relative cell recovery after cells underwent transduction according to workflow of FIG. 1 with TBK1/IKKε inhibitor MRT67307 HCl as a transduction aid.

FIG. 11 shows plots of the percent mNeonGreen positive cells and relative cell recovery after cells underwent transduction according to workflow of FIG. 1 with TBK1/IKKε inhibitor BAY-985 as a transduction aid.

FIG. 12 shows plots of the percent mNeonGreen positive cells and relative cell recovery after cells underwent transduction according to workflow of FIG. 1 with the TBK1/IKKε inhibitor GSK8612 on transduction efficiency.

FIG. 13 shows plots of the percent mNeonGreen positive cells and relative cell recovery after cells underwent transduction according to workflow of FIG. 1 with the TAK1 inhibitor 5Z-7-oxozeaenol as a transduction aid.

FIG. 14 shows plots of the percent mNeonGreen positive cells and relative cell recovery after cells underwent transduction according to workflow of FIG. 1 with the TBK1/IKKε inhibitor NG25 as a transduction aid.

FIG. 15 shows plots of the percent mNeonGreen positive cells and relative cell recovery after cells underwent transduction according to workflow of FIG. 1 with the TAK1 inhibitor takinib as a transduction aid.

FIG. 16 shows plots of the percent mNeonGreen positive cells and relative cell recovery after cells underwent transduction according to workflow of FIG. 1 with the TBK1/IKKε inhibitor HS-276 as a transduction aid.

FIG. 17 shows plots of the relative percent of mNeonGreen positive cells and relative cell recovery 6 hours and 24 hours post transduction using various combinations of F-108, BX795, and 5Z-7-oxozeaenol as transduction aids. Gray bars represent the best efficiency/recovery balanced conditions.

FIG. 18 shows plots of the relative percent of mNeonGreen positive cells and the relative number of cells expressing mNeonGreen 6 hours and 24 hours post transduction using various combinations of F-108, BX795, and 5Z-7-oxozeaenol as a transduction aid.

FIG. 19 shows representative flow cytometry traces 6 hours and 24 hours after transduction of the lentiviral vector of FIG. 1 using no aids (control) or F-108 as a transduction aid.

FIG. 20 shows representative flow cytometry traces 6 hours and 24 hours after transduction of the lentiviral vector of FIG. 1 using BX795 alone or BX795 and F-108 as a transduction aid.

FIG. 21 shows plots indicating the effect of cell density, plate bottom type (flat, rounded bottom (U), and conical bottom (V), and transduction volume on transduction efficiency for cells transduced with the lentiviral vector of FIG. 1 using BX795 and F-108 as transduction aids.

FIG. 22 shows the multiplicity of infection (MOI) titration curves of transduction efficiency for various transduction conditions.

FIG. 23 shows representative flow cytometry traces of FIG. 22 when the MOI was 5.

FIG. 24 shows the experimental workflow for testing transduction efficiency for transducing the lentiviral vector of FIG. 1 under cytokine-only stimulation conditions.

FIG. 25 shows plots of the percent of mNeonGreen positive cells after a high density (2×10⁶ cell/mL) of cells were transfected using BX795, F-108, and IL-2 or IL-5 as transduction aids and a low density of cells (0.5×10⁶ cell/mL) were transduced without BX795, F-108, and IL-2 or IL-5 as transduction.

FIG. 26 shows a plot of the percent of mNeonGreen positive cells after transduction with various combinations of different cytokines in addition to BX795 and F-108 transduction aids.

FIG. 27 shows the experimental workflow for testing transduction efficiency using BX797 and F-108 as transduction aids in various cell culture media (B0 medium, DMEM, RPMI, IMDM, NK XPANDER (Thermo Fisher Scientific, Waltham, MA) and NK MACS, Miltenyi Biotec B.V. and Co., Bergisch Gladbach, Germany) and with and without various transduction cell culture supplements (e.g., hAB, FBS, hPL, immune cell SR, CELL-VIVE Serum Substitute, BioLegend, San Diego, CA).

FIG. 28 shows a plot of the percent mNeonGreen positive cells after cells were transduced using BX797 and F-108 as transduction aids in various cell media.

FIG. 29 shows a plot of the percent of mNeonGreen positive cells after cells were transduced using BX797 and F-108 as transduction aids without or without the addition of various other transaction supplements.

FIG. 30 shows plots of the percent of mNeonGreen positive cells after cells were transduced using BX797 and F-108, RETRONECTIN, vectofusin-1, or various combinations thereof as transfection aids with or without spinfection (high speed centrifugation).

FIG. 31 shows a plot of the percent of mNeonGreen positive cells after cells were transduced with the lentiviral vector of FIG. 1 but with various different promoters and BX795 and F-108 transduction aids.

FIG. 32 shows a plot of the mean of fluorescent intensity (MFI) after cells were transduced with the lentiviral vector of FIG. 1 but with various different promoters and BX795 and F-108 transduction aids.

FIG. 33 shows the long-term tracking of CAR expression using CAG promoter. The usage of CAG promoter maintains transgene expression stability and strength over 4 weeks of expansion.

FIG. 34 shows a robust enhancement of lentiviral transduction across various therapeutic payload sizes. For lentiviral vector size 5047 bp, the transduction enhancer cocktail increased efficiency ~27-fold, from 2.22% to 59.1%. For lentiviral vector size 6337 bp, the transduction enhancer cocktail increased efficiency ~30-fold, from 1.07% to 31.7%.

FIG. 35 shows a robust enhancement of lentiviral transduction across various therapeutic payload sizes. For lentiviral vector size 6934 bp, the transduction enhancer cocktail increased the efficiency ~38-fold, from 0.86% to 32.5%. For lentiviral vector size 8041 bp, the transduction enhancer cocktail increased transduction efficiency ~250-fold, from 0.09% to 23.3%.

### DETAILED DESCRIPTION OF ILLUSTRATIVE EMBODIMENTS

The advance of cellular immunotherapy has revolutionized the field of cancer and autoimmune disease treatment. Genetic manipulation is a technique to unlock the therapeutic potential of immune effector cells, such as T or natural killer (NK) cells.

Among all the techniques, retroviral/lentiviral gene delivery has been shown to be safe and effective for ex vivo cellular product generation. Conventional retroviruses/lentiviruses are pseudo-typed with vesicular stomatitis virus G (VSV-G) glycoprotein envelopes, which can facilitate the entry of viral particles into target cells. For example, FDA-approved CAR-T cell products are manufactured using a VSV-G retrovirus or lentivirus. Recently, the genetic engineering of NK cells has gained much attention. However, VSV-G lentivirus transduction is generally ineffective in NK cells. Attempts to enhance the VSV-G lentivirus transduction in NK cells include various techniques that have not resulted in comparable transduction efficiency to T cells when using therapeutic-relevant constructs. For example, Senti Bio, a company focusing on genetic circuit-gated NK cell therapy, published an NK cell transduction method that uses a retronectin-coated plate and an hour-long high-speed centrifuge step, but still only got limited efficiency (Frankel et al. 2024 CellReport).

Various strategies to improve NK transduction efficiency have been pursued, including blocking the innate immune response TBK1/IKKε signaling pathway. VSV-G has been known to trigger type I interferon responses resulting in the dampening of transduction efficiency. Although the TBK1/IKKε inhibition may be effective, current methods include specific plate coating and time-consuming high-speed centrifuge steps. Another strategy being explored to increase NK cell transduction efficiency is changing the envelope from VSV-G to different pseudostyle. However, the use of different pseudotyped envelopes raises concerns about xeno-derived origin and low viral vector manufacturing capability. For example, the titer of RD 114-TR and BaEVRless pseudotyped viral vectors are 100-fold less than VSV-G pseudotyped viral vectors. Additionally, only VSV-G has been tested clinically and approved by the FDA. As such, a strategy for making VSV-G retrovirus/lentivirus transduction more efficient in NK cells is of high interest.

The present disclosure describes, in one aspect, a transduction composition. The transduction composition includes one or more transduction aids. Transduction aids can enhance transduction efficiency. In one or more embodiments, the transduction composition can be used to transduce a viral vector into a lymphocyte.

A transduction composition can be used before, during, or after transduction. Two or more transduction compositions may be used during a single transduction protocol. In some such embodiments, the two or more transduction compositions may be combined to form a single transduction composition that includes the components of the two or more transduction compositions.

A transduction composition includes one or more transduction aids. Transduction aids include polymers, enzyme inhibitors, cytokines or a cytokine activator, exogenous DNA components, or any combination thereof.

In one or more embodiments, a transduction composition includes a nonionic polymer, a cationic polymer, or both.

In one or more embodiments, a transduction composition includes a nonionic polymer. An example of a nonionic polymer is a poloxamer. Poloxamers are a class of triblock polymer having a hydrophobic poly(propylene oxide) (PPO) center block with a hydrophilic poly(ethylene oxide) (PEO) block on either side of the center block. Poloxamers can be denoted as PEOₐ-PPO_{b}-PEOₐ where a is the number of PEO repeat units (—CH₂CH₂O—) and b is the number of PPO (—CH(CH₃)—CH₂—O—) repeat units, and where a and b are averages and a can be from 2 to 130 and b can be from 15 to 70. Examples of poloxamers that could be included in a transduction composition include poloxamer F-108 (PEO₁₃₂-PPO₅₀-PEO₁₃₂), poloxamer 407 (PEO₁₀₁-PPO₅₆-PEO₁₀₁), poloxamer 338 (PEO₁₄₁-PPO₄₄-PEO₁₄₁), or any combination thereof. Poloxamers are commercially available. For example, poloxamer F-180, poloxamer 407, and poloxamer 338, are available from Millipore Sigma (St. Louis, MO).

In one or more embodiments, the transduction composition includes a poloxamer. Stated differently, in one or more embodiments, the transduction complex includes a poly(ethylene oxide)-poly(propylene oxide)-a poly(ethylene oxide) triblock polymer. In one or more embodiments, the transduction composition poloxamer F-108. In one or more embodiments, the transduction composition includes poloxamer 407. In one or more embodiments, the transduction composition includes poloxamer 338.

In one or more embodiments, a transduction composition includes a cationic polymer. Examples of a cationic polymers include diethylaminoethyl-dextran (DEAE-dextran), hexadimethrine bromide (polybrene), and protamine. In one or more embodiments, a transduction composition includes DEAE-dextran, a DEAE-dextran salt, a DEAE-dextran hydrate, or any combination thereof. In one or more embodiments, a transduction composition includes polybrene, a polybrene salt, a polybrene hydrate, or any combination thereof. In one or more embodiments, a transduction composition includes protamine, a protamine salt, a protamine hydrate, or any combination thereof.

In one or more embodiments, a transduction composition includes an enzyme inhibitor. The enzyme inhibitor may inhibit an enzyme of a signaling pathway. For example, an enzyme inhibitor may inhibit an immune response in a cell subject to transduction. Inhibition of the immune response may enhance transduction efficiency. For example, VSV-G (e.g., of VSV-G pseudotyped vectors) can trigger type 1 interferon responses. Production of type 1 interferons (cytokines) can be through the TANK-binding kinase 1 (TBK1) signaling pathway. Production of type 1 interferons (cytokines) can be through the TANK-binding kinase 1 (TBK1)/IκB kinase-ε (IKKε) dual signaling pathway. Inhibiting TBK1 and/or IKKε may inhibit or decrease the immune response of a host cell to the transduced vector. An enzyme inhibitor of mitogen-activated protein kinase kinase kinase 7 (MAP3K7), also known as TAK1, can decrease cytokine expression.

In one or more embodiments, the transduction composition includes a TBK1 inhibitor. Examples of TBK1 inhibitors that can be included in a transduction composition include TBK1 PROTACs and TBK1 molecular glue degraders. In one or more embodiments, the transduction composition includes a TBK1/IKKε inhibitor. Examples of TBK1/IKKε inhibitors that can be included in a transduction composition include BX795 (CAS No.: 702675-74-9), MRT67307 (CAS No.: 2095432-39-4), BAY-985 (CAS No.: 2409479-29-2), GSK8612 (CAS No. 2361659-62-1), amlexanox (CAS No. 68302-57-8), TBK1/IKKε—IN-1, TBK1/IKKε—IN-2, TBK1/IKKε —IN-4, TBK1/IKKε—IN-5, HPN-01, GSK319347A (CAS No. 862812-98-4) or any combination thereof. In one or more embodiments, a transduction complex includes BX795. In one or more embodiments, a transduction complex includes MRT67307. In one or more embodiments, a transduction complex includes BAY-985. In one or more embodiments, a transduction complex includes GSK8612.

In one or more embodiments, the transduction composition includes a TAK1 inhibitor. Examples of TAK1 inhibitors that can be included in a transduction composition include 5Z-7-oxozeaenol (CAS No.: 253863-19-3), NG25 (CAS No.: 1315355-93-1), takinib (CAS No.: 1111556-37-6), HS-276 (CAS No.: 2767422-72-8), dehydroabietic acid (CAS No. 1740-19-8), sarsasapogenin (CAS No. 26-19-2), TAK1-IN-4 (CAS No. 1570374-32-1), PF-05381941 (CAS No. 1474022-02-0), INH14 (CAS No. 200134-22-1), HS-243 (CAS No. 848249-10-5), TAK-756, BPD, IRAK4-IN-21 (CAS No. 2170694-04-7), IRAK4-IN-22 (CAS No. 2170694-05-8), 4-hydroxywogonin (CAS No. 57096-02-3), 1,3,5-trihydroxy-4-prenylxanthone (CAS No. 53377-61-0), TAK-715 (CAS No. 303162-79-0), or any combination thereof. In one or more embodiments, a transduction complex includes 5Z-7-oxozeaenol. In one or more embodiments, a transduction complex includes NG25. In one or more embodiments, a transduction complex includes takinib. In one or more embodiments, a transduction complex includes HS-276.

In one or more embodiments, a transduction composition includes a cytokine. Some cytokines induce cell proliferation, such as, for example interleukin 2 (IL-2), interleukin (Il-12), interleukin-15 (IL-15), interlueikin18 (IL-18), interleukin 21 (IL-21). As such, activating endogenous cytokine production in a cell via a cytokine activator, or treating cells with exogenous cytokines may increase transduction efficiency.

In one or more embodiments, a transduction composition includes a cytokine. In one or more embodiments, a transduction composition includes IL-2, IL-4, IL-6, IL-12, IL-15, IL-18, IL-21, IFN-gamma, G-CSF, GM-CSF, or any combination thereof. In one or more embodiments, a transduction composition includes IL-2. In one or more embodiments, a transduction composition includes IL-12. In one or more embodiments, a transduction composition includes IL-15. In one or more embodiments, a transduction composition includes IL-18. In one or more embodiments, a transduction composition includes IL-21.

In one or more embodiments, a transduction composition includes a cytokine in an amount of 1 ng/mL to 100 ng/mL. In one or more embodiments, a transduction composition includes a cytokine in an amount of 1 ng/mL or greater, 5 ng/mL or greater, 10 ng/mL or greater, 15 ng/mL or greater, 20 ng/mL or greater, 30 ng/mL or greater, 40 ng/mL or greater, 50 ng/mL or greater, 60 ng/mL or greater, 70 ng/mL or greater, 80 ng/mL or greater, or 90 ng/mL or greater. In one or more embodiments, a transduction composition includes a cytokine in an amount of 100 ng/mL or less, 90 ng/mL or less, 80 ng/mL or less, 70 or ng/mL or less, 60 ng/mL or less, 50 ng/mL or less, 40 ng/mL or less, 30 ng/mL or less, 20 ng/mL or less, 15 ng/mL or less, 10 ng/mL or less, or 5 ng/mL or less. In one or more embodiments, a transduction composition includes a cytokine in an amount of 1 ng/mL to 30 ng/mL.

A transduction composition that includes a cytokine can be used, for example, to activate a cell or cells. As such, transduction composition that includes a cytokine can be incubated with a cell or cells prior to exposing the cell to the viral vector to be transduced.

In one or more embodiments, a transduction composition includes exogenous DNA components. Examples of DNA components that may be included in transduction composition include nucleobases, deoxynucleosides, and deoxynucleotides. In one or more embodiments, a transduction composition includes nucleobase. In one or more embodiments, a transduction composition includes deoxynucleosides. In one or more embodiments, a transduction composition includes deoxynucleotides.

In one or more embodiments, a transduction composition includes a nonionic polymer and an enzyme inhibitor. In one or more embodiments, a transduction composition includes a poloxamer and an TBK1/IKKε inhibitor. In one or more embodiments, a transduction composition includes a poloxamer F-108 and BX795.

In one or more embodiments, a transduction composition includes a nonionic polymer and a cytokine or a cytokine activator. In one or more embodiments, a transduction composition includes a poloxamer and a cytokine or a cytokine activator. In one or more embodiments, a transduction composition includes a poloxamer F-108 and a cytokine or a cytokine activator.

In one or more embodiments, a transduction composition includes a viral vector. Viral vectors can transfer genetic material into a host cell through the natural system of viral infection and multiplication. In one or more embodiments, a transduction composition includes an adeno-assisted viral vectors, lentiviral vectors, adenoviral vectors, retroviral vectors, herpes simplex viral vectors, Epstein-Bar viral vectors, hybrid adenoviral vectors, or any combination thereof. In one or more embodiments, a transduction composition includes a lentiviral vector. In one or more embodiments, a transduction composition includes a VSV-G pseudotyped lentiviral vector.

The viral vector includes a promoter. The promoter of a viral vector may be any suitable promoter. Example promoters include, but are not limited to, CMV, EFS, EF1a, MND, PGK, RSV, SFFV, SV40, and UBC. In one or more embodiments, the viral vector includes the CMV promoter. In one or more embodiments, the viral vector includes the EFS promoter. In one or more embodiments, the viral vector includes the EF1a promoter. In one or more embodiments, the viral vector includes the MND promoter. In one or more embodiments, the viral vector includes the PGK promoter. In one or more embodiments, the viral vector includes the RSV promoter. In one or more embodiments, the viral vector includes the SFFV promoter. In one or more embodiments, the viral vector includes the SV40 promoter. In one or more embodiments, the viral vector includes the UBC promoter.

A transduction composition may further include a medium. A transduction composition may include any suitable cell culture medium. Example cell culture media include B0 (DMEM/F-12 (2:1) supplemented with 10% human AB serum, 20 μM 2-mercaptoethanol, 50 μM ethanolamine, 20 μg/mL ascorbic acid, 5 ng/mL sodium selenite, and 100 U/mL penicillin/streptomycin), Dulbecco's modified eagle medium (DMEM), RPMI 1640 medium (Roswell Park Memorial Institute), Iscove's modified Dulbecco's medium (IMDM), NK-EXPANDER medium, NK-MACS medium, CELLGENIX GMP SCGM, EXCELLERATE human NK cell expansion media, Click's medium or any combination thereof. In one or more embodiments, a transduction composition includes B0 medium. In one or more embodiments, a transduction composition includes DMEM. In one or more embodiments, a transduction composition includes RPMI medium. In one or more embodiments, a transduction composition includes IMDM. In one or more embodiments, a transduction composition includes NK-EXPANDER medium. In one or more embodiments, a transduction composition includes NK-MACS medium.

In one or more embodiments, a transduction composition may include one or more additives. Example additives include serum and/or serum replacements or substitutes such as, for example, hAB, FBS, immune cell serum replacement, and CELLVIVE serum substitute.

In one or more embodiments, a transduction composition includes a cell or a plurality of cells (sometimes just called cells). The cell or plurality of cells can be lymphocytes. The lymphocytes can be T cells, B cells, or a natural killer (NK) cells. In one or more embodiments, a transduction composition includes a lymphocyte or lymphocytes. In one or more embodiments, a transduction composition includes an NK cell or NK cells.

Each transduction aid present in a transduction composition can be provided in an amount effective to enhance transduction of a vector into a cell. An effective amount of a transduction aid is the amount of the transduction aid that increases transduction efficiency by 1-fold or greater, 2-fold or greater, 3-fold or greater, 4-fold or greater, 5-fold or greater, 6-fold or greater, 7-fold or greater 8-fold or greater, 9-fold or greater, or 10-fold or greater compared to the same transduction composition lacking the transduction aid.

The absolute amount of a transduction aid in a transduction composition can vary. For example, in one or more embodiments, a transduction composition includes 0.01 mg/mL to 100 mg/mL of a polymer. In one or more embodiments, a transduction composition includes 0.01 mg/mL of greater, 1 mg/mL or greater, 2 mg/mL or greater, 5 mg/mL or greater, 10 mg/mL or greater, 15 mg/mL or greater, 20 mg/mL or greater, 30 mg/mL or greater, 40 mg/mL or greater, 50 mg/mL or greater, 60 mg/mL or greater, 70 mg/mL or greater, 80 mg/mL or greater, or 90 mg/mL or greater of a polymer. In one or more embodiments, a transduction composition includes 100 mg/mL or less, 90 mg/mL or less, 80 mg/mL or less, 70 mg/mL or less, 60 mg/mL or less, 50 mg/mL or less, 40 mg/mL or less, 30 mg/mL or less, 20 mg/mL or less, 15 mg/mL or less, 10 mg/mL or less, 9 mg/mL or less, 8 mg/mL or less, 7 mg/mL or less, 6 mg/mL or less, 5 mg/mL or less, 4 mg/mL or less, 3 mg/mL or less, 2 mg/mL or less, 1 mg/mL or less, or 0.1 mg/mL or less of a polymer. In one or more embodiments, a transduction composition includes 0.1 mg/mL to 10 mg/mL of a polymer.

In one or more embodiments, a transduction composition includes 0.01 μM to 100 μM of an enzyme inhibitor. In one or more embodiments, a transduction composition includes 0.01 μM of greater, 1 μM or greater, 2 μM or greater, 5 μM or greater, 10 μM or greater, 15 μM or greater, 20 μM or greater, 30 μM or greater, 40 μM or greater, 50 μM or greater, 60 μM or greater, 70 μM or greater, 80 μM or greater, or 90 μM or greater of an enzyme inhibitor. In one or more embodiments, a transduction composition includes 100 μM or less, 90 μM or less, 80 μM or less, 70 μM or less, 60 μM or less, 50 μM or less, 40 μM or less, 30 μM or less, 20 μM or less, 15 μM or less, 10 μM or less, 9 μM or less, 8 μM or less, 7 μM or less, 6 μM or less, 5 μM or less, 4 μM or less, 3 μM or less, 2 μM or less, 1 μM or less, or 0.1 μM or less of an enzyme inhibitor. In one or more embodiments, a transduction composition includes 1 μM to 8 μM of an enzyme inhibitor.

In one or more embodiments, a transduction composition includes the virus vector in an amount from 1×10⁵ transduction unit (TU) per 1×10⁶ cells to 1×10⁸ TU per 1×10⁶ cells. In one or more embodiments, a transduction composition includes the virus vector in an amount from 1×10⁵ TU or greater per 1×10⁶ cells, 2.5×10⁵ TU or greater per 1×10⁶ cells, 5×10⁵ TU or greater per 1×10⁶ cells, 7.5×10⁵ TU or greater per 1×10⁶ cells, 1×10⁶ TU or greater per 1×10⁶ cells, 2.5×10⁶ TU or greater per 1×10⁶ cells, 5×10⁶ TU or greater per 1×10⁶ cells, 7.5×10⁶ TU or greater per 1×10⁶ cells, 1×10⁷ TU or greater per 1×10⁶ cells, 2.5×10⁷ TU or greater per 1×10⁶ cells, 5×10⁷ TU or greater per 1×10⁶ cells, or 7.5×10⁷ TU or greater per 1×10⁶ cells. In one or more embodiments, a transduction composition includes the virus vector in an amount from 1×10⁸ TU or less per 1×10⁶ cells, 7.5×10⁷ TU or less per 1×10⁶ cells, 5×10⁷ TU or less per 1×10⁶ cells, 2.5×10⁷ TU or less per 1×10⁶ cells, 1×10⁷ TU or less per 1×10⁶ cells, 7.5×10⁶ TU or less per 1×10⁶ cells, 5×10⁶ TU or less per 1×10⁶ cells, 2.5×10⁶ TU or less per 1×10⁶ cells, 1×10⁶ TU or less per 1×10⁶ cells, 7.5×10⁵ TU or less per 1×10⁶ cells, 5×10⁵ TU or less per 1×10⁶ cells, or 2.5×10⁵ TU or less per 1×10⁶ cells. In one or more embodiments, a transduction composition includes the virus vector in an amount from 5×10⁵ to 5×10⁷ TU per 1×10⁶ cells.

In one or more embodiments, a transduction composition has a multiplicity of infection (MOI; the ratio of viral particles to target cells) of 0.1 to 100. In one or more embodiments, a transduction composition has an MOI of 0.1 or greater, 0.5 or greater, 1 or greater, 5 or greater, 10 or greater, 15 or greater, 20 or greater, 30 or greater, 40 or greater, 50 or greater, 60 or greater, 70 or greater, 80 or greater, or 90 or greater. In one or more embodiments, a transduction composition has an MOI of 100 or less, 90 or less, 80 or less, 70 or less, 60 or less, 50 or less, 40 or less, 30 or less, 20 or less, 15 or less, 10 or less, 5 or less, 1 or less, or 0.5 or less. In one or more embodiments, a transduction composition has an MOI of 0.1 to 50.

A high density (2×10⁶ cell/mL) of cells were transfected using BX795, F-108, and IL-2 or IL-5 as transduction aids and a low density of cells (0.5×10⁶ cell/mL).

In one or more embodiments, a transduction composition includes 1×10⁵ cells/mL to 1×10⁷ cells/mL. In one or more embodiments, a transduction composition includes 1×10⁵ cells or more, 2.5×10⁵ cells/mL or more, 5×10⁵ cells/mL or more, 7.5×10⁵ cells/mL or more, 0.5×10⁶ cells/mL or more, 1×10⁶ cells/mL or more, 2.5×10⁶ cells/mL or more, 5×10⁶ cells/mL or more, 7.5×10⁶ cells/mL or more, 1×10⁷ cells/mL or more, 2.5×10⁷ cells/mL or more, 5×10⁷ cells/mL or more, 7.5×10⁷ cells/mL or more. In one or more embodiments, a transduction composition includes 1×10⁸ cells/mL or less, 7.5×10⁷ cells/mL or less, 5×10⁷ cells/mL or less, 2.5×10⁷ cells/mL or less, 1×10⁷ cells/mL or less, 7.5×10⁶ cells/mL or less, 5×10⁶ cells/mL or less, 2.5×10⁸ cells/mL or less, 1×10⁶ cells/mL or less, 0.5×10⁶ cells/mL or less, 7.5×10⁵ cells/mL or less, 5×10⁵ cells/mL or less, or 2.5×10⁵ cells/mL or less. In one or more embodiments, a transduction composition includes 0.5×10⁶ cell/mL to 2 10⁶ cell/mL.

In another aspect, the present disclosure describes a transduction method. In one or more embodiments, the transduction method does not include a centrifugation step between virus addition and media exchange.

The transduction method includes contacting a cell with transduction composition. In one or more embodiments, the method includes contacting a plurality of cells (or just cells) with a transduction composition. The transduction composition can be a transduction composition disclosed herein. In one or more embodiments, the cell or the cells are lymphocytes. In one or more embodiments, the cell or the cells are NK cells.

In one or more embodiments, the method includes incubating the cell or cells with the transduction composition for a transduction time. The transduction time can vary. In one or more embodiments, the transduction time is 10 min to 84 hours. In one or more embodiments, the transduction time is 10 min or greater, 1 hour or greater, 12 hours or greater, 24 hours or greater, 32 hours or greater, 48 hours or greater, 60 hours or greater, or 72 hours or greater. In one or more embodiments, the transduction time is 84 hours or less, 72 hours or less, 60 hours or less, 48 hours or less, 32 hours or less, 24 hours or less, 12 hours or less, or 1 hour or less. In one or more embodiments, the transduction time is 6 hours to 24 hours. In one or more embodiments, the transduction time is 60 hours to 84 hours.

In one or more embodiments, the method includes washing the cell or cells. Washing the cell or cells can remove excess components of the transduction composition. Washing can be used, for example, to quench transduction and/or reduce overall cell loss.

In one or more embodiments, the method includes expanding the cell or cells. In one or more embodiments, expanding the cell or cells includes incubating the cell or cells with feeder cells. Examples of a feeder cells include Gy irradiated mbIL21-41BBL K562 cells and mbIL15-41BBL K562 cells. In one or more embodiments, expanding the cell or cells does not include incubating with feeder cells. Examples of feeder cell free activation or expansion methods and kits include NK Cell Activation/Expansion Kit (anti-NKp46, CD2 antibody-conjugated magnetic beads), GROW-NK (anti-NKp46, CD2 antibody complex), and Cloudz Human NK Cell Expansion Kit (anti-NKp46, CD2 antibody-loaded microsphere).

In one or more embodiments, the method includes contacting a cell or cells with a first transduction composition that includes a cytokine for an activating time and contacting the cell or cells with a second transduction composition that includes at least the viral vector for a transduction time. In one or more embodiments, the first transduction composition includes one or more cytokines. In one or more embodiments, the activating time can be 60 hours to 84 hours.

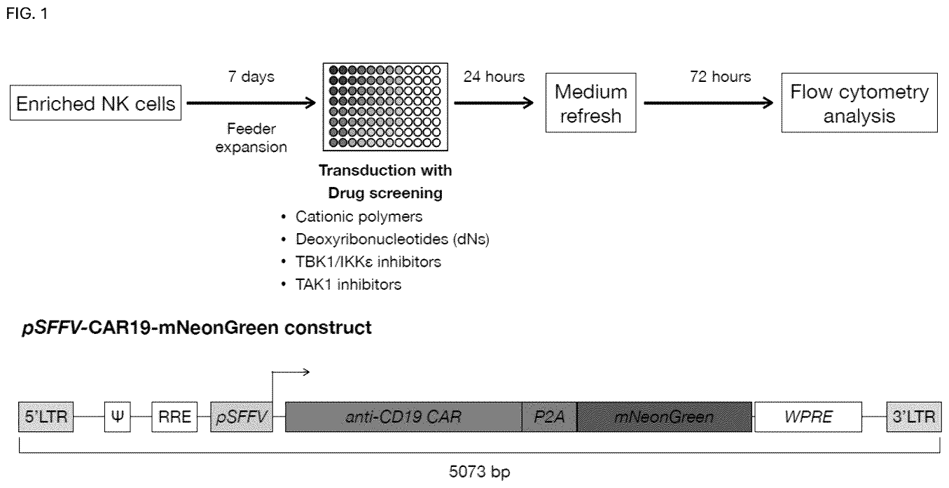

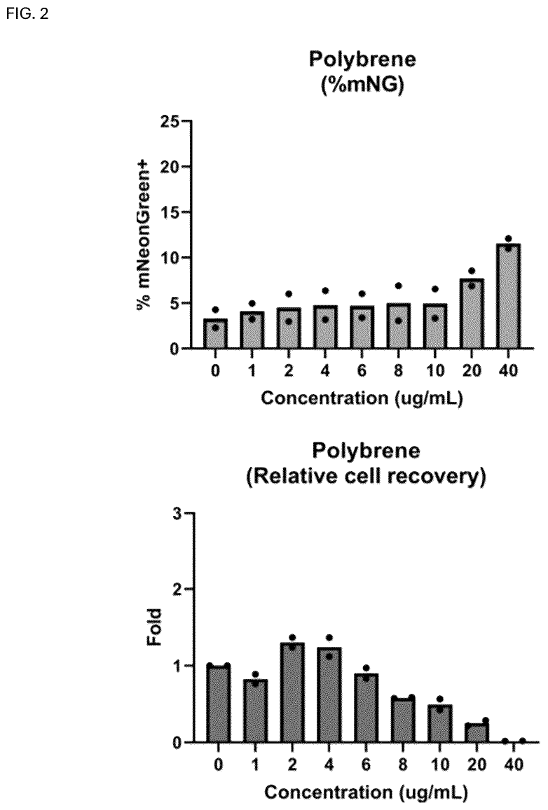

Various molecules were screened for their ability to facilitate the VSV-G lentivirus transduction in NK cells. FIG. 1 shows the experimental workflow for the transduction aid screening. Briefly, enriched NK cells were isolated from PBMC and allowed to expand with irradiated mbIL21-41BBL K562 feeder cells for 7 days before transduction of the lentiviral vector shown in FIG. 2. For transduction, the compounds shown in Table 1 were screened by inclusion in the transduction media with the lentiviral vector for 24 hours. After 24 hours, the medium was refreshed, and NK cells were cultured for another 72 hours before flow cytometry analysis. FIG. 2 shows the construct design for CAR-NK generation. The second-generation lentiviral vector contains a self-inactivating (SIN) HIV genome. The vector encodes anti-CD19 CAR with mNeonGreen reporter driven by SFFV promoter. The size of the lentiviral genome was 5073 bp. Table 1 shows the molecules that were screened, the concentrations of the molecules screened, and the figures in which the results are shown.

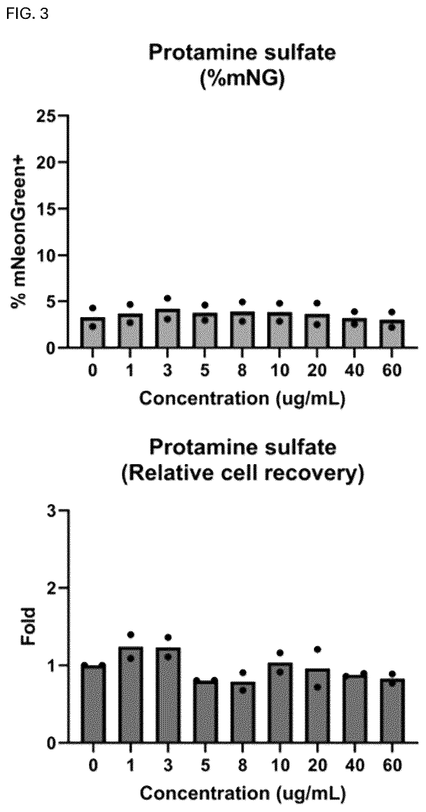

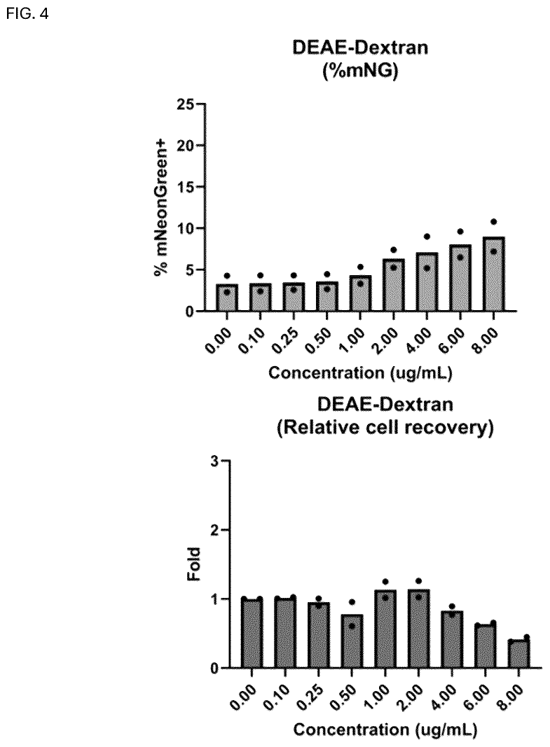

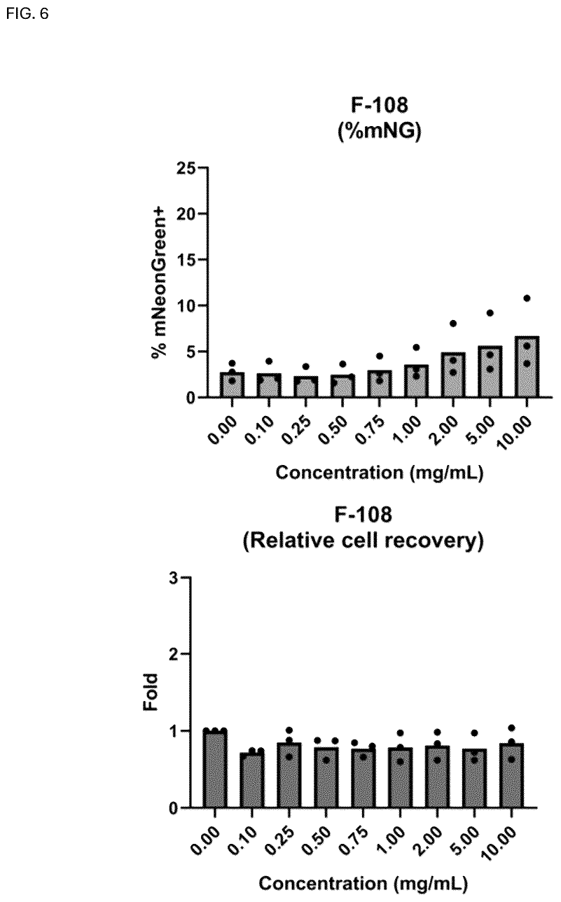

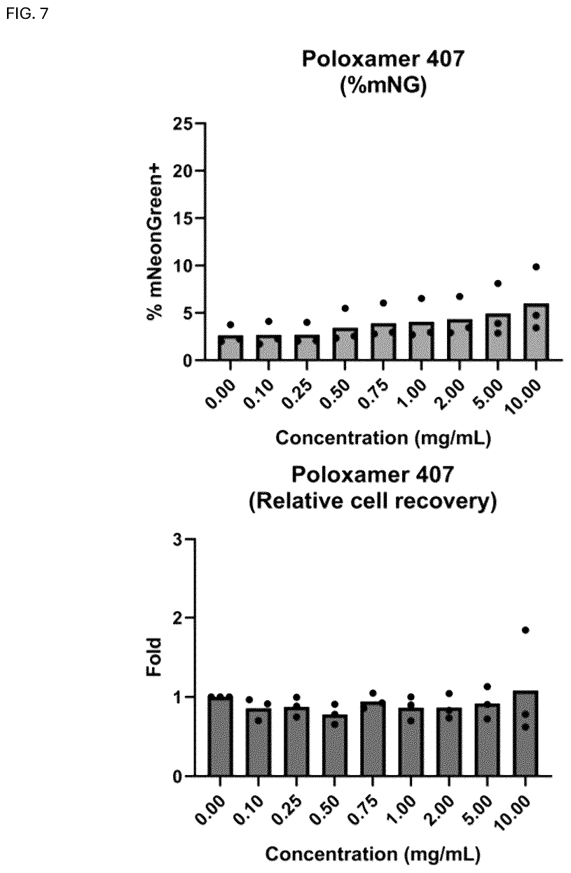

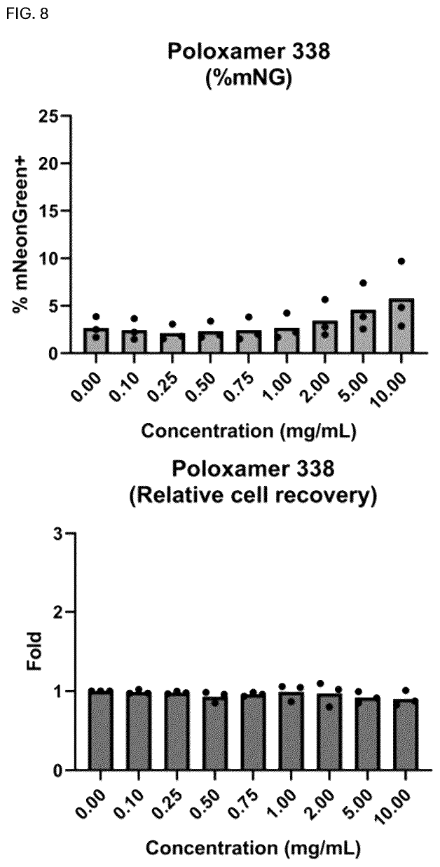

The effect of the polymers screened varied. Cationic polymers polybrene and DEAE-Dextran boosted transduction efficiency but led to low cell recovery after transduction (FIG. 2 and FIG. 4). Protamine sulfate did not increase the transduction efficiency (FIG. 3). The nonionic polymers screened, poloxamer F-108, poloxamer 407, and poloxamer 338, all enhanced the transduction efficiency while maintaining a good cell recovery after transduction (FIG. 6, FIG. 7, and FIG. 8).

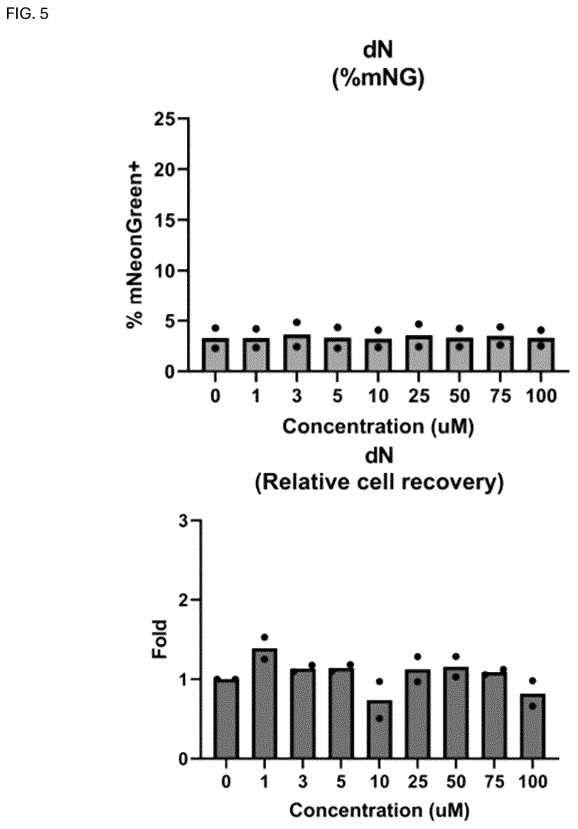

The inclusion of dNs did not increase transduction efficiency (FIG. 5).

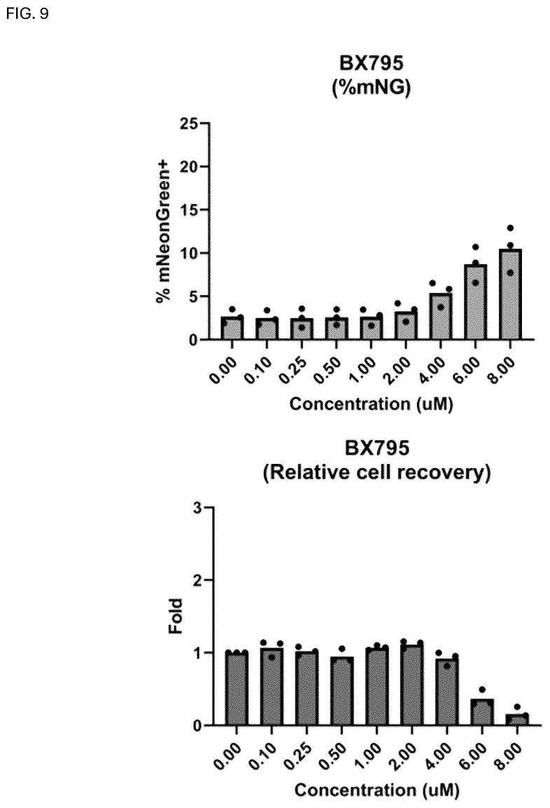

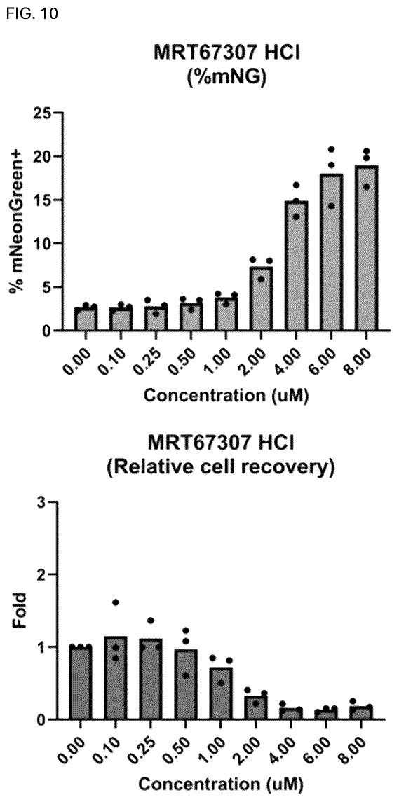

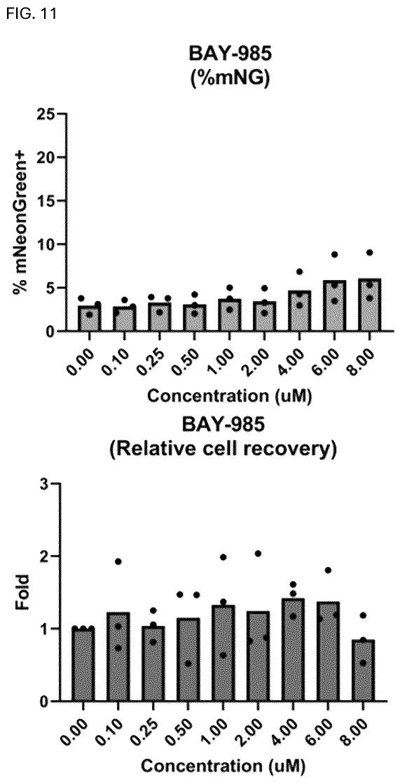

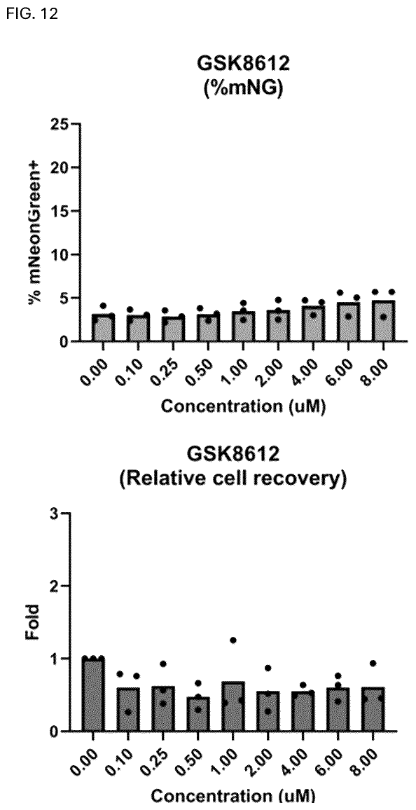

The effect of the TBK1/IKKε inhibitors screened varied. BX795 and MRT67307 increased the transduction efficiency but had reduced the cell recover when used at higher concentrations (FIG. 9 and FIG. 10). BAY-985 was able to generate higher efficiency but not as effective as BX795 and MRT67307 (FIG. 11). GSK8612 did not show increased transduction efficiency (FIG. 12).

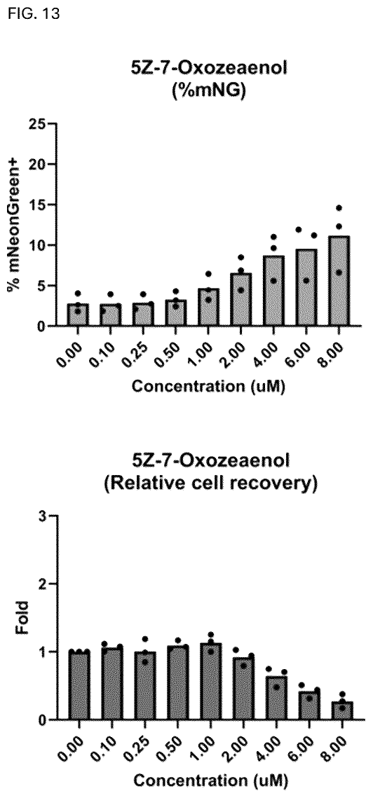

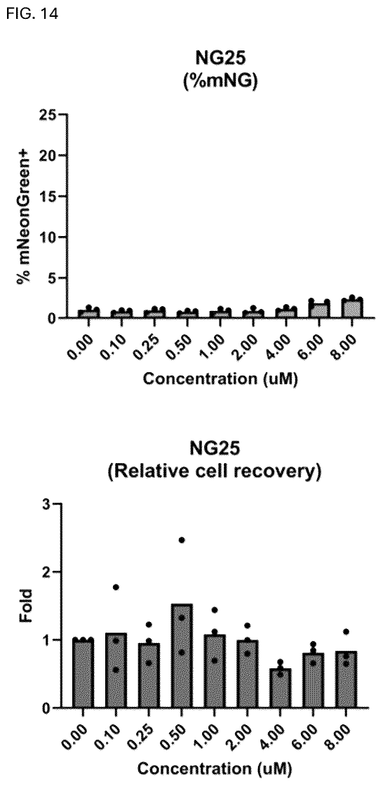

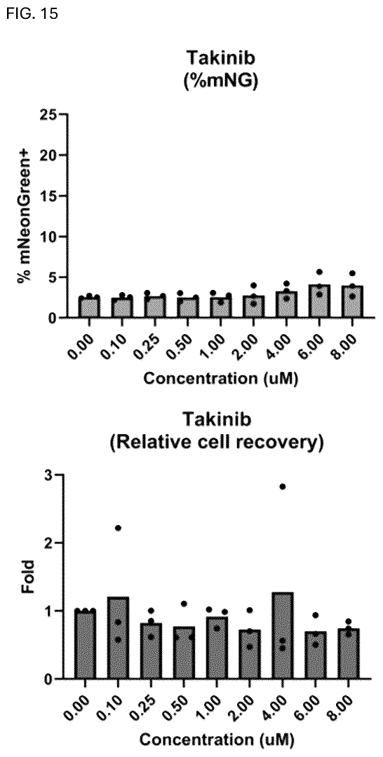

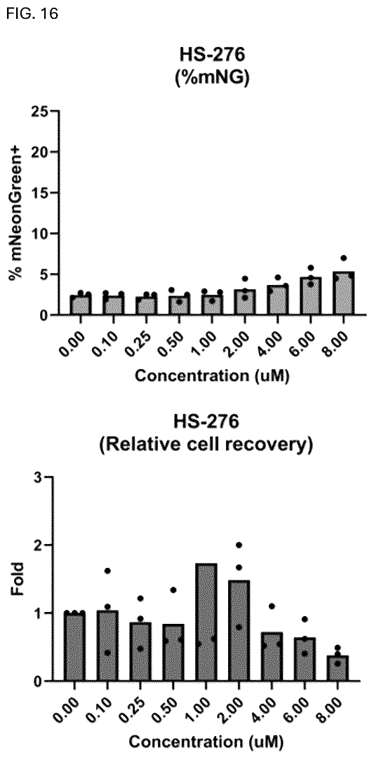

The effect of the TAK1 inhibitors screened varied. 5Z-7-oxozeaenol was able to boost the transduction efficiency in a dose-dependent manner with reduced cell recovery (FIG. 13). TAK1 inhibitors NG25 and takinib did not enhance the transduction efficiency (FIG. 14 and FIG. 15). HS-276 showed a slight increase the transduction efficiency but the cell recovery dropped (FIG. 16).

TABLE 1

| Molecules | | Results |
|---|---|---|
| Screened | Concentrations screened | Shown |
| polybrene | 0, 1, 2, 4, 6, 8, 10, 20, and 40 μg/mL | FIG. 2 |
| protamine | 0, 1, 3, 5, 8, 10, 20, 40, and 60 μg/mL | FIG. 3 |
| sulfate | | |
| DEAE-dextran | 0.00, 0.10, 0.25, 0.5, 1.0, 2.0, 4.0, 6.0, | FIG. 4 |
| | and 8.0 μg/mL | |
| dN | 0, 1, 3, 5, 10, 25, 50, 75, 100 μM | FIG. 5 |
| Poloxamer | 0, 0.1, 0.25, 0.5, 0.75, 1, 2, 5, and 10 mg/mL | FIG. 6 |
| F-108 | | |
| Poloxamer 407 | 0, 0.1, 0.25, 0.5, 0.75, 1, 2, 5, and 10 mg/mL | FIG. 7 |
| Poloxamer 338 | 0, 0.1, 0.25, 0.5, 0.75, 1, 2, 5, and 10 mg/mL | FIG. 8 |
| BX795 | 0, 0.1, 0.25, 0.5, 1, 2, 4, 6, and 8 μM | FIG. 9 |
| MRT67307 | 0, 0.1, 0.25, 0.5, 1, 2, 4, 6, and 8 μM | FIG. 10 |
| HCl | | |
| BAY-985 | 0, 0.1, 0.25, 0.5, 1, 2, 4, 6, and 8 μM | FIG. 11 |
| GSK8612 | 0, 0.1, 0.25, 0.5, 1, 2, 4, 6, and 8 μM | FIG. 12 |
| 5Z-7- | 0, 0.1, 0.25, 0.5, 1, 2, 4, 6, and 8 μM | FIG. 13 |
| oxozeaenol | | |
| NG25 | 0, 0.1, 0.25, 0.5, 1, 2, 4, 6, and 8 μM | FIG. 14 |
| Takinib | 0, 0.1, 0.25, 0.5, 1, 2, 4, 6, and 8 μM | FIG. 15 |
| HS-276 | 0, 0.1, 0.25, 0.5, 1, 2, 4, 6, and 8 μM | FIG. 16 |

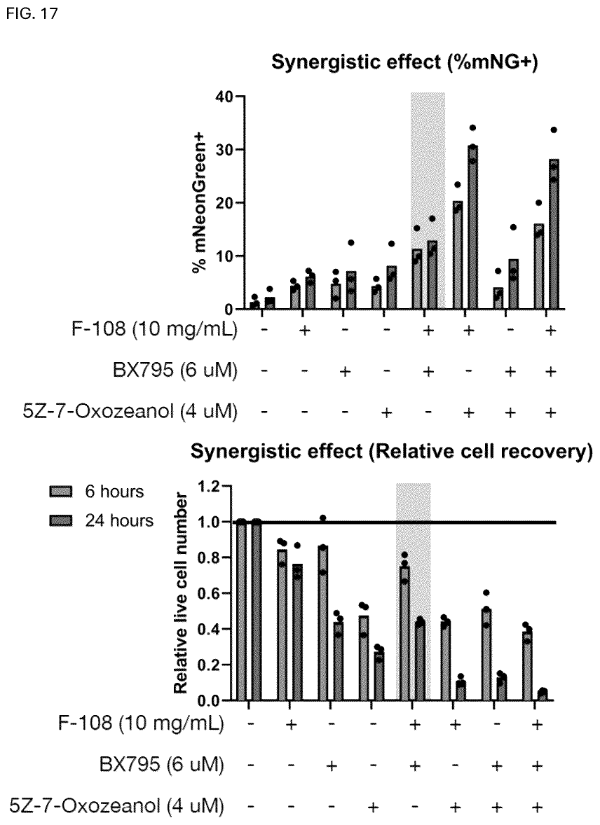

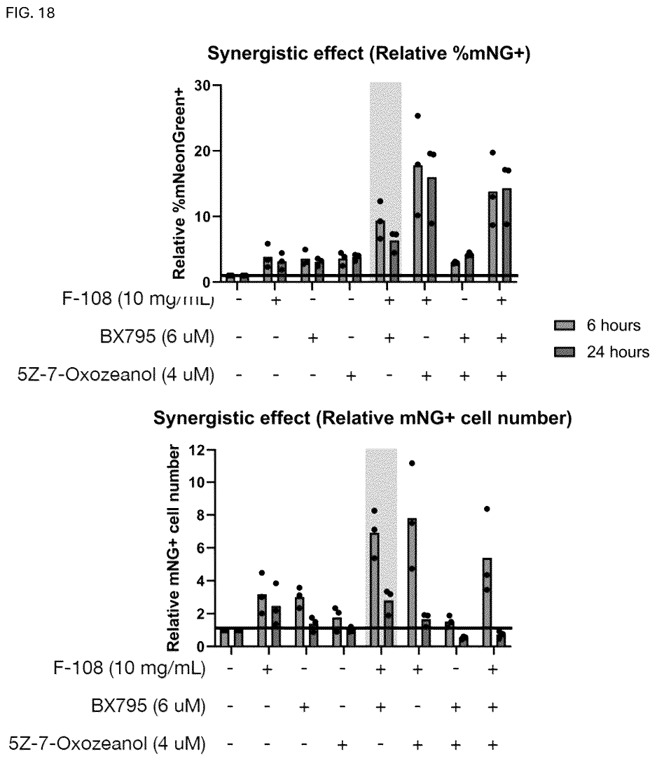

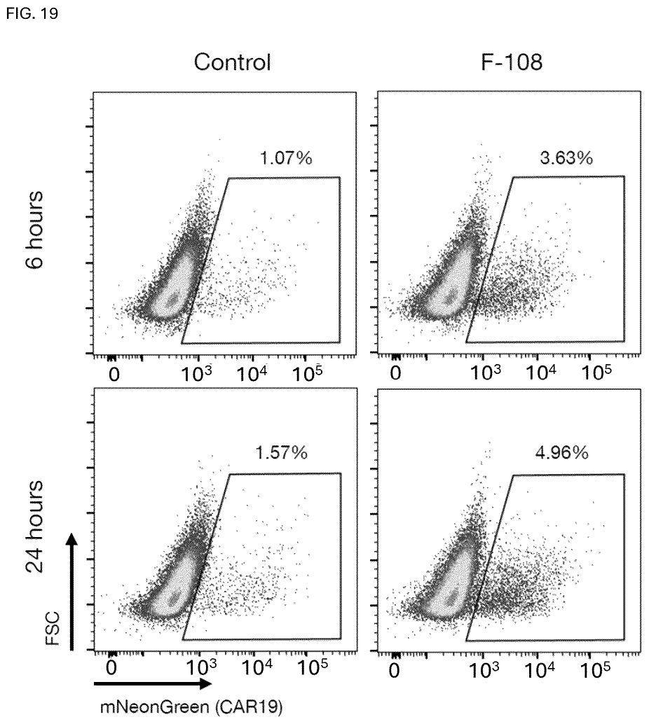

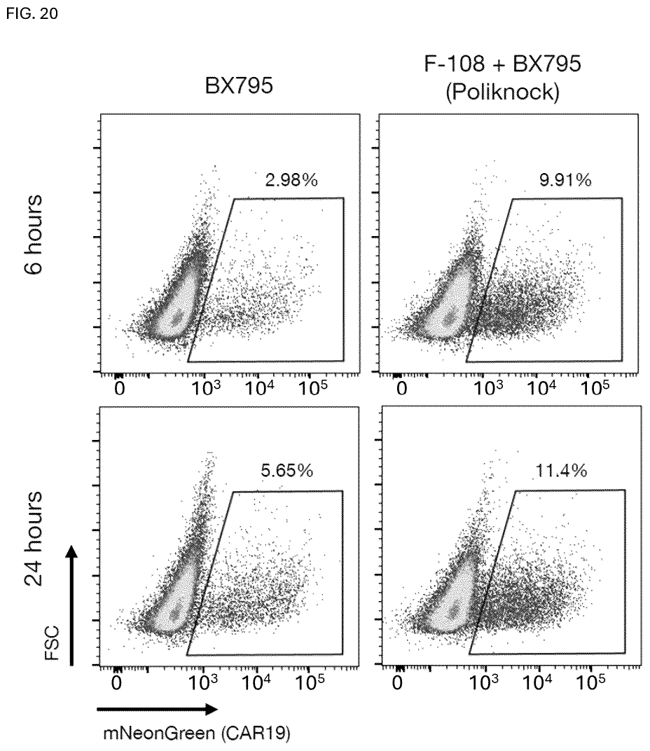

Following the individual screening of various compounds, combinations of poloxamer F-108, BX795, and 5Z-7oxozeaenol were screened. FIG. 17 and FIG. 18 show the results and FIG. 19 and FIG. 20 show representative flow cytometry traces for the combination screening. Each of the compounds alone was able to increase the transduction efficiency. When F-108 was combined with BX795 or 5Z-7-oxozeaenol, the synergistic effect was able to further improve the transduction efficiency from 1% to 12%. Co-inhibition of TBK1/IKKε and TAK1 by BX795 and 5Z-7-Oxozeanol was unable to provide benefits. Six hours of transduction retained the transduction enhancement effects while maintaining around 75% cell recovery using the F-108 and BX795 combination. The F-108 and BX795 combination is also referred to as poliknock.

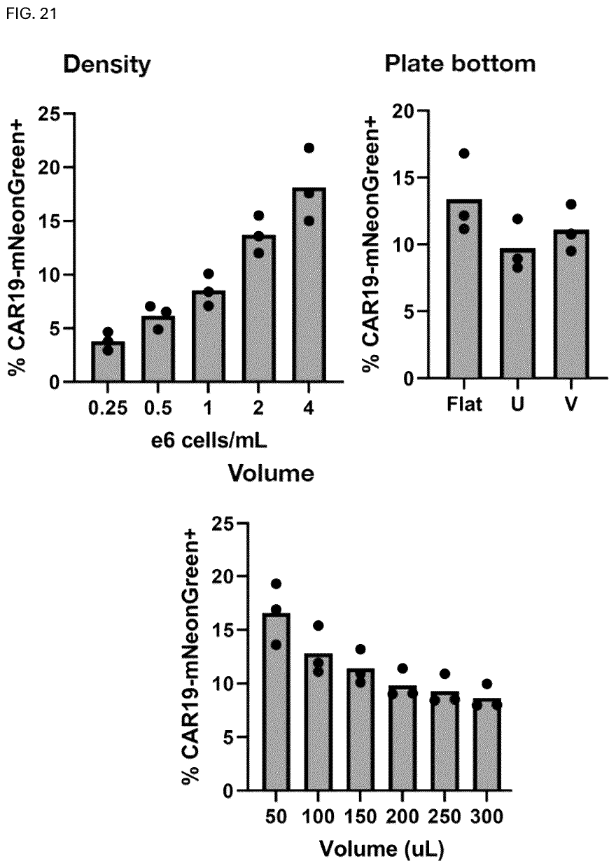

The F-108 and BX795 transduction aid combination (poliknock) was tested in different culture settings (FIG. 21). The transduction efficiency increased when cell density was raised. When using a 96-well plate, a flat bottom provided the highest transduction efficiency. Smaller transduction volume was beneficial to transduction efficiency.

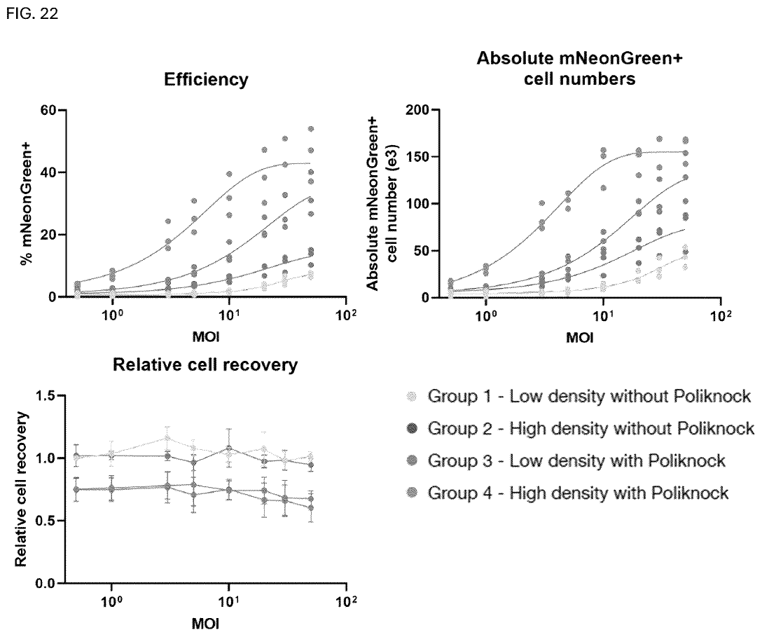

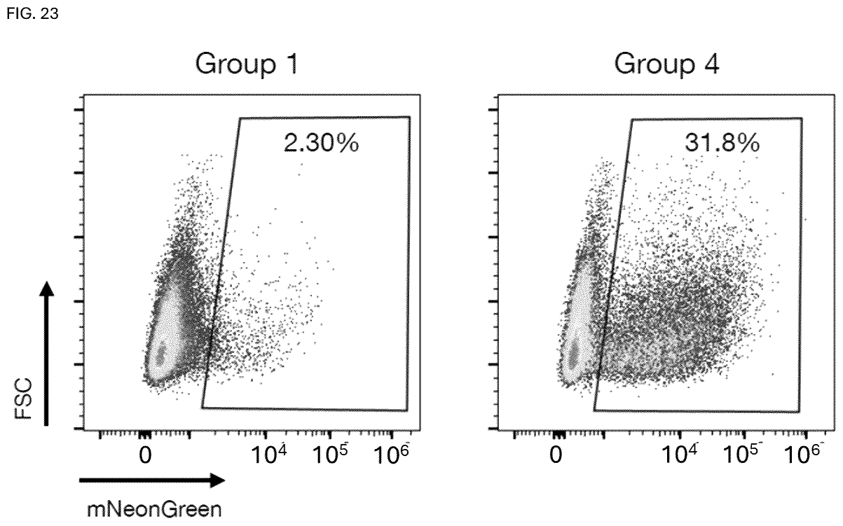

FIG. 22 shows multiplicity of infection (MOI) titration curves of transduction efficiency for the F-108 and BX795 transduction aid combination with low and high cell density compared to transduction without the F-108 and BX795 combination. Poliknock boosted transduction efficiency independent of cell density. When combined with high-density culture, poliknock resulted in the highest transduction efficiency and the enhancement across different MOI. The efficiency increased from less than 1% to more than 20% when the MOI was 5. At a high MOI of 20, the efficiency increased from 3% to 40%. Poliknock with high density transduction was able to produce more than 100K transduced cells. Cell loss during transduction is mainly from the toxicity of Poliknock, instead of elevated MOI. FIG. 23 is the representative flow cytometry traces for when the MOI was 5.

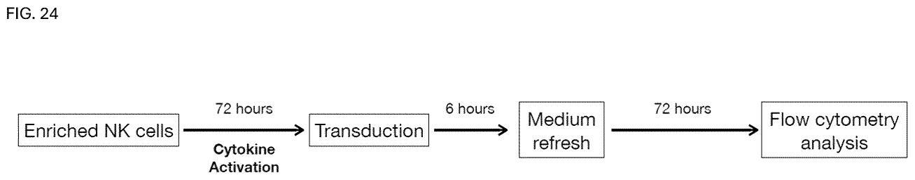

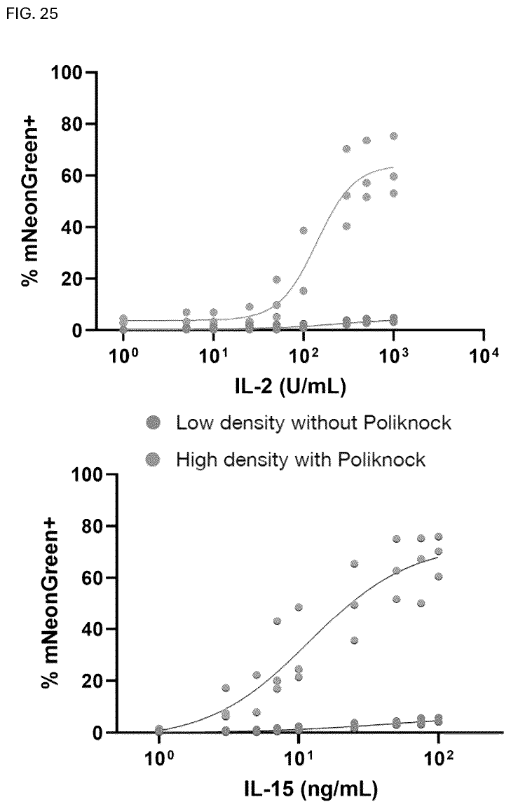

The transduction efficiency of the F-108 and BX795 transduction aid combination was tested in combination with IL-2 or IL-5 cytokines following the experimental workflow shown in FIG. 24. Specifically, various concentrations of IL-2 or IL-5 were incubated with the cells prior to transduction. Poliknock boosted transduction efficiency in NK cells treated with IL-2 or IL-15 in a concentration-dependent manner (FIG. 25). When the MOI was 5, the efficiency increased from 3% to 60% in a high IL-2 concentration culture. High IL-15 concentration showed similar results.

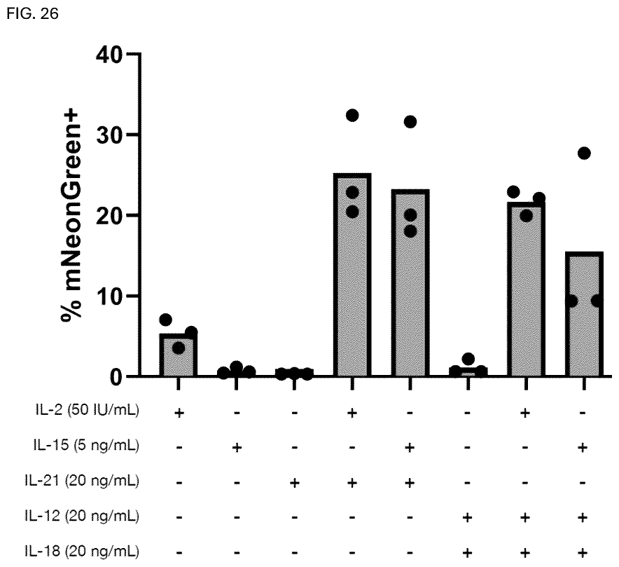

FIG. 26 shows the synergistic effect of different cytokines that can lower the need for IL-2 and IL-15 concentration following the experimental workflow of FIG. 24. Briefly, enriched cells were incubated with various cytokines prior to transduction using the F-108 and BX795 transduction aid combination. When combined with IL-21 or IL-12/18, a low dosage of IL-2 or IL-15 could enhance transduction efficiency. IL-21 and IL-12/18 alone were unable to generate meaningful transduction efficiency.

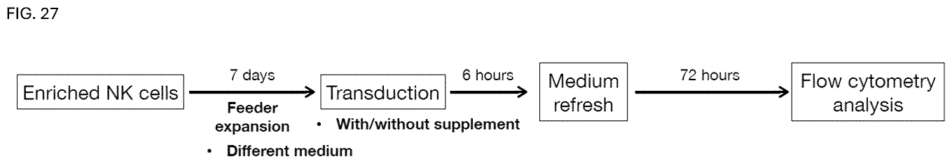

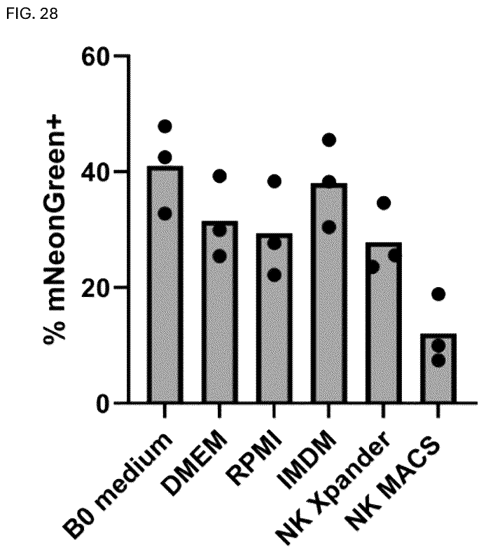

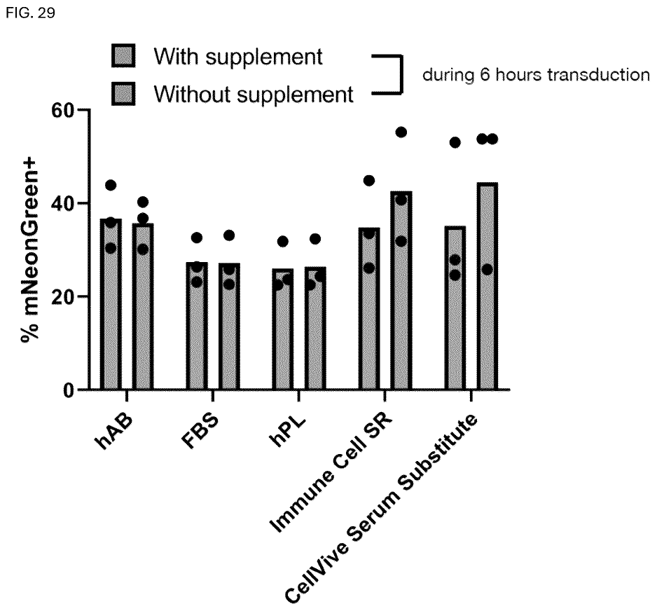

The transduction efficiency of the F-108 and BX795 transduction aid combination was tested with various media and additives using experimental workflow shown in FIG. 27. The transduction efficiency of poliknock was robust across different cell culture mediums (FIG. 28). Additionally, presence of different cell culture supplements during 6 hours of transduction did not affect the benefit of poliknock (FIG. 29).

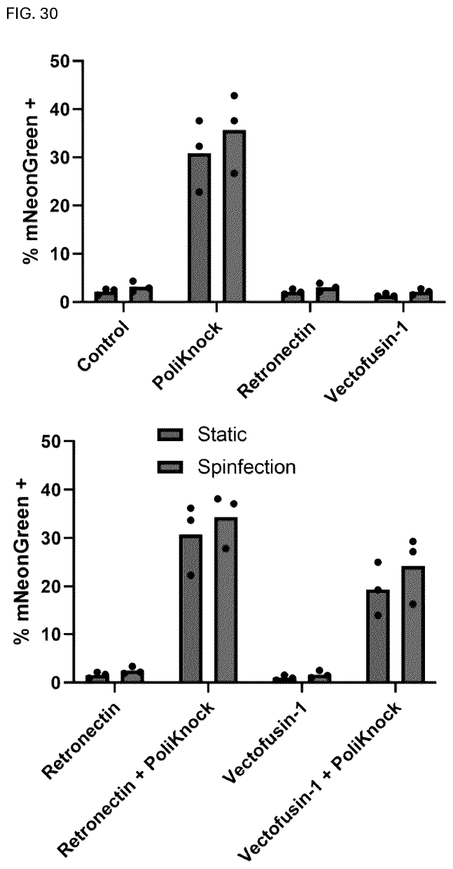

The transduction efficiency of the F-108 and BX795 transduction aid was compared to the conventional transduction aids retronectin and vectofusin-1 with and without the use of high speed centrifuge (spinfection). Additional poliknock was testing in combination with retronectin and vectofusin-1. Retronectin and vectofusin-1 did not provide a meaningful transduction efficiency boost (FIG. 30). Additionally, no synergistic effect between poliknock with retronectin or poliknock with vectofusin-1, and this was spinfection-independent (FIG. 30).

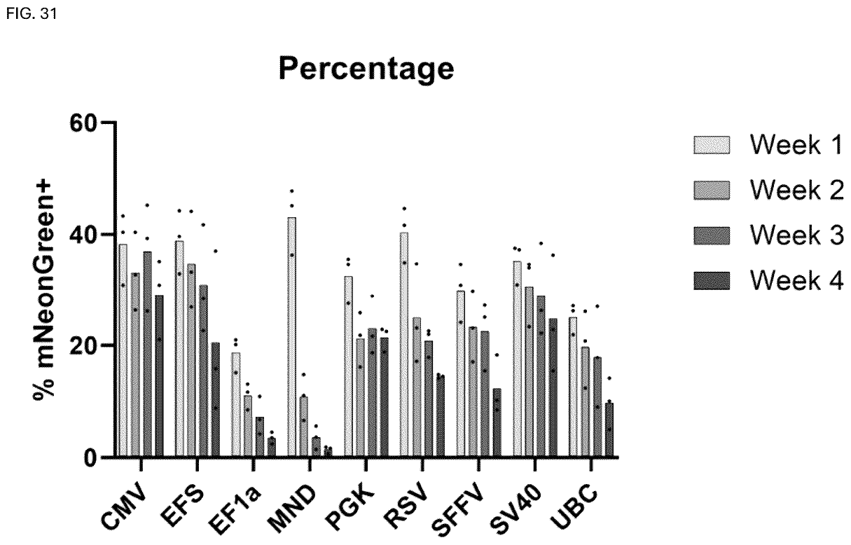

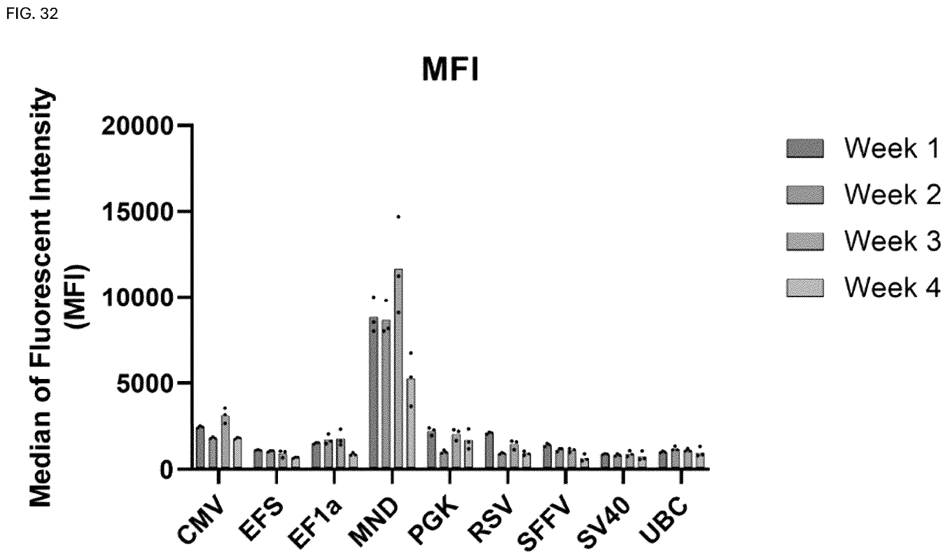

The transduction efficiency of the F-108 and BX795 transduction aid combination was tested with the same lentiviral vector as shown in FIG. 2 but with different promoters. The transduction yielded high percentage of transduced population, but they dropped after several rounds of feeder expansion (FIG. 31 and FIG. 32). For example, the MND promoter generated the highest transduced population, but the promoter activity was silenced quickly after just one round of feeder expansion. CMV, EFS, PGK, and SV40 maintained a high frequency of mNeonGreen positive population. Several rounds of expansion didn't cause dramatically reduction of median of fluorescence intensity (MFI) over time (FIG. 32).

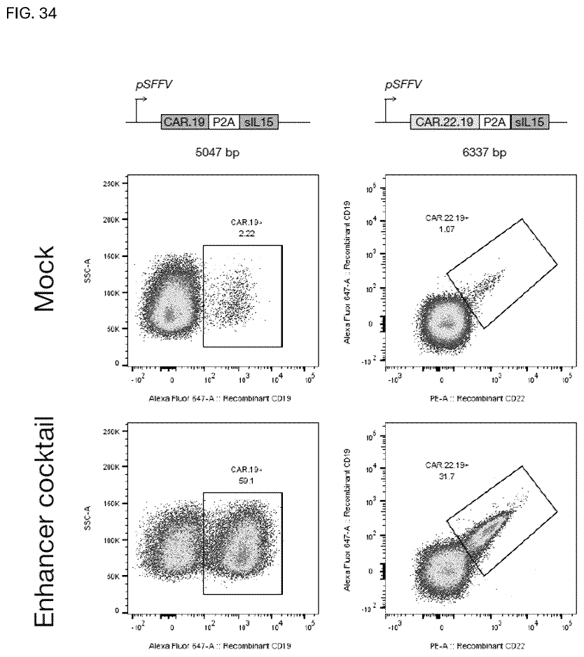

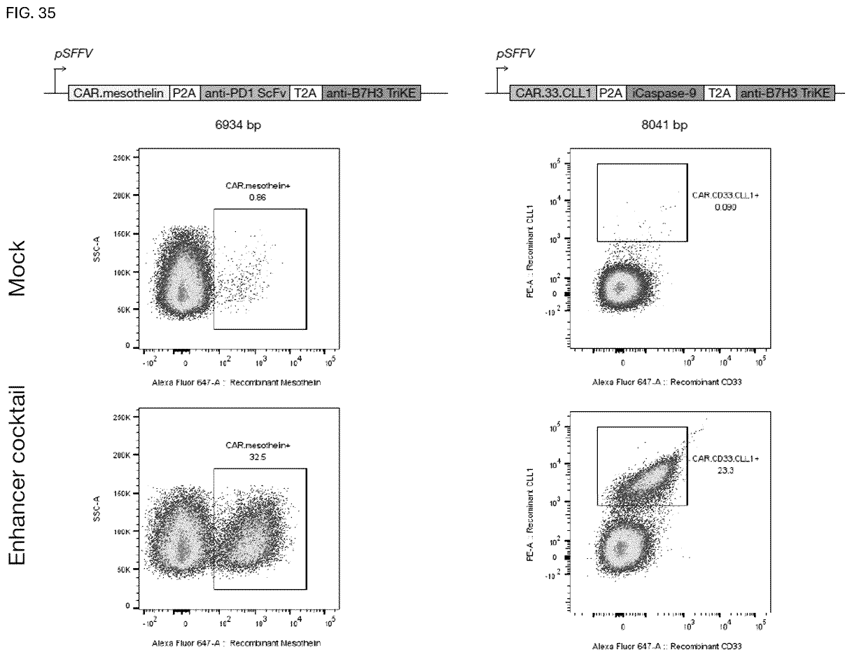

Robust enhancement of lentiviral transduction was demonstrated across various therapeutic payload sizes (FIG. 34 and FIG. 35). For lentiviral vector size 5047 bp, the transduction enhancer cocktail increased efficiency ~27-fold, from 2.22% to 59.1%. For lentiviral vector size 6337 bp, the transduction enhancer cocktail increased efficiency ~30-fold, from 1.07% to 31.7%. For lentiviral vector size 6934 bp, the transduction enhancer cocktail increased the efficiency ~38-fold, from 0.86% to 32.5%. For lentiviral vector size 8041 bp, the transduction enhancer cocktail increased transduction efficiency ~250-fold, from 0.09% to 23.3%.

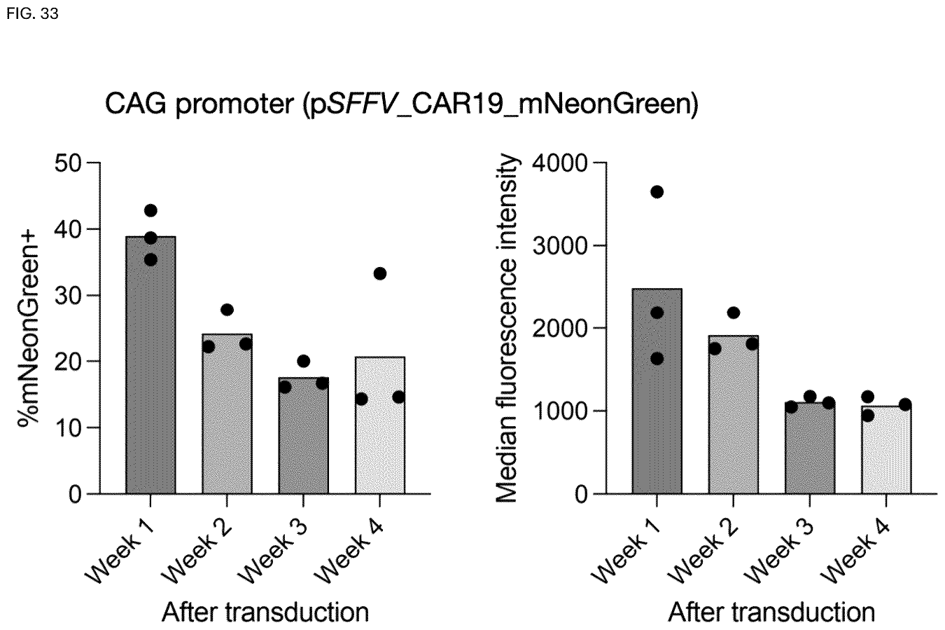

FIG. 33 shows the long-term tracking of CAR expression using CAG promoter. The usage of CAG promoter maintains transgene expression stability and strength over 4 weeks of expansion.

In the preceding description and following claims, the term “and/or” means one or all of the listed elements or a combination of any two or more of the listed elements; the terms “comprises,” “comprising,” and variations thereof are to be construed as open ended i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

As used herein, “have,” “has,” “having,” “include,” “includes,” “including,” “comprise,” “comprises,” “comprising” or the like are used in their open-ended inclusive sense, and generally mean “include, but not limited to,” “includes, but not limited to,” or “including, but not limited to.” Further, wherever embodiments are described herein with the language “have,” “has,” “having,” “include,” “includes,” “including,” “comprise,” “comprises,” “comprising” and the like, otherwise analogous embodiments described in terms of “consisting of” and/or “consisting essentially of” are also provided. The term “consisting of” means including, and limited to, that which follows the phrase “consisting of.” That is, “consisting of” indicates that the listed elements are required or mandatory, and that no other elements may be present. The term “consisting essentially of” indicates that any elements listed after the phrase are included, and that other elements than those listed may be included provided that those elements do not interfere with or contribute to the activity or action specified in the disclosure for the listed elements.

As used herein, the word “exemplary” means to serve as an illustrative example and should not be construed as preferred or advantageous over other embodiments.

As used herein, the terms “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

In the preceding description, particular embodiments may be described in isolation for clarity. Reference throughout this specification to “one embodiment,” “an embodiment,” “certain embodiments,” “one or more embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

In several places throughout the above description, guidance is provided through lists of examples, which examples can be used in various combinations. In each instance, the recited list serves only as a representative group and should not be interpreted as an exclusive list.

For any method disclosed herein that includes discrete steps, the steps may be performed in any feasible order. And, as appropriate, any combination of two or more steps may be performed simultaneously.

### EXAMPLES

The present disclosure is illustrated by the following examples. It is to be understood that the particular examples, materials, amounts, and procedures are to be interpreted broadly in accordance with the scope and spirit of the invention as set forth herein.

### Isolation, Culture, and Expansion of Human Primary NK Cells:

Human peripheral blood mononuclear cells (PBMC) were isolated from healthy donor fresh blood using FICOLL (GE Healthcare, Chicago, IL) density centrifugation. Human primary NK cells were isolated from PBMC using Human NK Cell Enrichment Kit (STEMCELL Technologies, Inc., Vancouver, BC) as per manufacturer instructions. For the first round of feeder expansion, isolated NK cells were expanded by 100 Gy irradiated mbIL21-41BBL K562 feeder cells at 1:2 NK:feeder ratio in either IMDM (Thermo Fisher Scientific, Inc., Waltham, MA) with 10% human AB serum or B0 medium (DMEM/F-12 (2:1) supplemented with 10% human AB serum, 20 μM 2-mercaptoethanol, 50 μM ethanolamine, 20 μg/mL ascorbic acid, 5 ng/mL sodium selenite, and 100 U/mL penicillin/streptomycin) with 50 IU/mL recombinant human IL-2, unless otherwise stated. The medium doubling and fresh IL-2 supplement was performed on day 3 and day 5. Day 7 was the final day of each round of feeder expansion. Starting from second round, the NK:feeder ratio was adjusted to 1:1, followed by protocol aforementioned.

### Plasmid Cloning:

All plasmid cloning was conducted using isothermal reaction by NEBuilder® HiFi DNA Assembly (New England Biolabs, Ipswich, MA). All gene fragments were chemically synthesized (Twist Biosciences, San Francisco, CA). The mNeonGreen protein sequence was derived from Shaner et al, 2013 (PMID: 23524392). The CAR19 protein sequence was derived from Zah et al, 2016 (PMID: 27059623). The CAR.22.19, CAR.mesothelin, CAR.33.CLL1, anti-PD-1 ScFv, iCaspase-9, and anti-B7H3 TriKE sequences were derived from WO 2026/005947 A1. The second-generation lentiviral vector was derived from pZR112_Lenti-SFFV-mCherry-2A-dCas9-VP64 (Cat. #180263, Addgene, Watertown, MA). All protein sequence was codon-optimized by GENSMART Codon Optimization Tool (GenScript, Piscataway, NJ). The assembled plasmids were transformed into NEB Stable competent cells (New England Biolabs, Ipswich, MA). All plasmids were sequence-verified by nanopore-based sequencing (Azenta Life Sciences, Burlington, MA).

### Lentivirus Production:

Lentivirus production was performed using second-generation lentiviral packaging plasmids—pMD2.G and psPAX2 (Addgene #12259 and #12260). Lenti-X 293T (Takara Bio, San Jose, CA) cells were maintained in DMEM (high glucose) supplemented with 1 mM sodium pyruvate, 1× GlutaMAX, 1×non-essential amino acid, and 25 mM HEPES. HT-1080 (ATCC, Manassas, VA) cells were maintained in DMEM (high glucose) supplemented with 10% heat-inactivated FBS. The lentivirus packaging medium was Opti-MEM supplemented with 5% heat-inactivated FBS, 1 mM sodium pyruvate, 1×GlutaMAX, 1×non-essential amino acid, and 25 mM HEPES. All cell culture reagents were purchased from ThermoFisher, unless otherwise stated. For lentivirus production, the day before transfection, 7×10⁶ Lenti-X 293T cells were resuspended in 12 mL of lentivirus packaging medium and seeded into a 10-cm cell culture plate. After 18-24 hours, 6 mL of the cell culture medium was removed and proceed to Lipofectamine 3000 transfection. 8 μg of psPAX2, 4 μg of pMD2.G, and 8 μg of transfer vector were co-transfected into Lenti-X 293T cells as per the manufacture's instruction. After 6 hours, the medium was completely removed and replaced with 12 mL of lentivirus packaging medium supplemented with 1×ViralBoost reagent (ALSTEM, Richmond, CA). The next day, 12 mL of fresh lentivirus packaging medium supplemented with 1× ViralBoost reagent was used to refresh the cell culture and allow continuous lentivirus production. The virus-containing medium was collected by centrifugation at 2000×g for 10 minutes to remove cell debris and stored at 4° C. until next day. Twenty-four hours later, the virus-containing medium was collected and pooled with the previously harvested medium. The pooled virus-containing medium was then concentrated 100× with Lenti-X concentrator as per the manufacture's instruction. The final virus stock was formulated in Opti-MEM and stored at −80° C. until use.

### Flow Cytometry Protocol:

Transduced NK cells were resuspended and transferred to a U-bottom 96-well plate. NK cells were pelleted at 400×g for three minutes, followed by PBS wash. After PBS wash, NK cells were stained with 50 μL of 1× Zombie Violet fixable viobility dye (BioLegend, San Diego, CA) diluted in PBS at room temperature in the dark for 15 min. Immediately after staining, 150 μL of FABS buffer (PBS supplemented with 1% human serum and 0.5 M EDTA) was added into each well to quench the staining process. Stained NK cells were pelleted at 400×g for three minutes. Pelleted NK cells were resuspended in 150 μL of FACS buffer containing 1 μL of CountBright Absolute Counting Beads (Thermo Fisher Scientific, Waltham, MA) for cell number quantification as per manufacture's suggestion.

### Transduction Aid Screening Protocol:

Day 7 expanded NK cells were resuspended at 2×10⁶ cells/mL in B0 medium. 50 μL of each transduction aid was pre-diluted and seeded into a flat-bottom 96-well plate. 25 μL of virus-containing medium (1×10⁶ TU) was then added into each well to achieve an MOI of 10.25 μL of resuspend NK cells were then added and mixed well. The transduction was allowed to proceed for 24 hours (transduction time) at 37° C. The next day, the transduction medium was removed by centrifugation and the cell pellet was resuspended in 200 μL of B0 medium supplemented with 50 IU/mL IL-2. The transduced NK cells were allowed to rest for another 48 hours before flow cytometry analysis.

### F-108, BX795, and 5Z-7-Oxozeaenol Combination Testing:

Various combinations of F-108, BX795, and 5Z-7-oxozeaenol combination were tested largely according to the transduction aid screening protocol. The transduction time was either 6 hours or 24 hours. For the 6-hour transduction condition, NK cells were pelleted and transferred into fresh B0 medium supplemented with 50 IU/mL after incubation with indicated concertation of transductions aids.

### Cell Density Testing, Plate Type Testing, and Transduction Volume Testing:

Cell density testing, plate type testing, and transduction volume testing was accomplished largely according to the transduction aid screening protocol where the BX795 and F-108 were used as transduction aids. For cell density testing, the plate format was a flat bottom, the transduction volume was 100 μL, and the cell density was varied. For plate format test, the cell concentration was 5×10⁵ cells/mL, the transduction volume was 100 μL, and the plate type was varied. For transduction volume test, the cell concentration was 5×10⁵ cells/mL, the plate format was flat bottom, and the transduction volume was varied.

### Transduction Protocol for MOI Testing; Cytokine Testing; Media Testing; Additive Testing; Vectofusin-1, Retronectin Testing and Centrifugation Testing; and Promoter Testing:

Day 7 expanded NK cells were resuspended at 4×10⁶ cells/mL in B0 medium. 50 μL of medium containing 20 mg/mL F-108 and 12 μM of BX795 was seeded into a flat-bottom 96-well plate. 25 μL of virus-containing medium was then added into each well to achieve indicated MOI. 25 μL of resuspend NK cells were then added and mixed well. The transduction was allowed to proceed for 6 hours at 37° C. After 6 hours, the transduction medium was removed by centrifugation and the cell pellet was resuspended in 200 μL of medium supplemented with 50 IU/mL IL-2. The transduced NK cells were allowed to rest for another 54 hours before flow cytometry analysis.

### MOI Testing:

Four groups were tested (group 1, group 2, group 3, and group 4). Groups 1 and 3 used 5×10⁵ cells/mL (high density) and groups 2 and 4 used 2×10⁶cells/mL (low density). Groups 3 and 4 included 20 mg/mL F-108 and 12 μM of BX795 was seeded into a flat-bottom 96-well plate.

### Cytokine Testing:

Enriched NK cells were activated in cytokine containing B0 medium for 72 hour prior to transduction.

Media testing:

Enriched NK cells were feeder-expanded in indicated medium supplemented with 10% human AB serum and 50 IU/mL IL-2.

### Additive Testing:

IMDM supplemented with indicated supplement with 50 IU/mL IL-2 were used for feeder-expansion.

### Vectofisin-1, Retronectin Testing, and Centrifugation Testing:

For retronectin and vectofusin-1, the manufacture's manual protocol was used. Briefly, for retronectin, a 96-well flat bottom plate was coated with 100 μL of 50 μg/mL retronectin per well at 4° C. overnight. After coating, the plate was washed three times with PBS. For vectofusin-1, the vectofusin-1 was mixed with virus to the final concentration of 20 μg/mL, then mixed with 1:1 volume with cell suspension to achieve final concentration of 10 μg/mL, followed by direct plating on a 96-well flat bottom plate. The transduction was either static (no centrifugation with a 6-hour incubation period) or dynamic (centrifugation, also called injection was used). For the dynamic transduction, the 6-hour static incubation period was replaced with centrifugation at 1200×g for 90 minutes.

### Promoter Testing:

The pSFFV promoter of the lentivirus shown in FIG. 1 was replaced with various other promoters.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A transduction composition comprising:
a poloxamer; and
a TBK1 inhibitor, a TAK1 inhibitor, or both.

2. The transduction composition of claim 1, wherein the poloxamer and the TBK1 inhibitor are provided in an amount effective to increase the transduction efficiency of a viral vector into a cell by 1-fold or greater compared to the same transduction composition lacking the poloxamer and the TBK1 inhibitor.

3. The transduction composition of claim 1, wherein the poloxamer comprises poloxamer F-108, poloxamer 407, or poloxamer 338.

4. The transduction composition of claim 1, wherein the TBK1 inhibitor comprises BX795, MRT67307, BAY-985, or GSK8612.

5. The transduction composition of claim 1, wherein the poloxamer comprises poloxamer F-108 and the TBK1 inhibitor comprises BX795.

6. The transduction composition of claim 1, wherein the TAK1 inhibitor comprises 5Z-7-oxozeaenol, NG25, or takinib.

7. The transduction composition of claim 1, wherein the poloxamer comprises F-108 and the TBK1 inhibitor comprises BX795.

8. The transduction composition of claim 1, wherein the transduction composition comprises 0.1 mg/mL to 10 mg/mL of the poloxamer.

9. The transduction composition of claim 1, wherein the transduction composition comprises 1 μM to 8 μM of the TBK1 inhibitor or TAK1 inhibitor.

10. The transduction composition of claim 1, wherein the transduction composition further comprises a viral vector.

11. The transduction composition of claim 10, wherein the viral vector comprises a lentiviral vector.

12. The transduction composition of claim 10, wherein viral vector is a VSV-G pseudotyped vector.

13. The transduction composition of claim 10, wherein the viral vector comprises a promoter and wherein the promoter comprises a CMV promoter, an EFS promoter, an EF1a promoter, an MND promoter, a PGK promoter, an RSV promoter, an SFFV promoter, a CAG promoter, or a UBC promoter.

14. The transduction composition of claim 1, wherein the transduction composition further comprises a cytokine.

15. The transduction composition of claim 14, wherein the cytokine comprises IL-2, IL-12, IL-15, IL-18, IL-21, or any combination thereof.

16. The transduction composition of claim 1, wherein the transduction composition further comprises a medium.

17. The transduction composition of claim 1, wherein the transduction composition further comprises a cell.

18. A transduction method comprising:
contacting a cell with the transduction composition of claim 1.

19. The method of claim 18, wherein the cell is a lymphocyte.

20. The method of claim 19, wherein the lymphocyte is a natural killer cell.
